(19) United States
(12) Patent Application Publication
(10) Pub. No.: US 2023/0088420 A1
(43) Pub. Date: Mar. 23, 2023

(54) METHOD FOR PRODUCTION OF LACTOBACILLUS PLANTARUM

(71) Applicant: Asahi Biocycle Co., Ltd., Tokyo (JP)

(72) Inventors: Gentaro YASUDA, Peachtree City, GA (US); Kotoyo TANAKA, Peachtree City, GA (US)

(73) Assignee: Asahi Biocycle Co., Ltd., Tokyo (JP)

(21) Appl. No.: 17/949,763

(22) Filed: Sep. 21, 2022

Related U.S. Application Data
(60) Provisional application No. 63/247,107, filed on Sep. 22, 2021.

(51) Int. Cl. C12N 1/20

(52) U.S. Cl. CPC C12N 1/205; C12R 2001/25

(57) ABSTRACT

This application provides a method for culturing Lactobacillus plantarum, which comprises culturing Lactobacillus plantarum in a medium comprising molasses, a yeast extract and sucrose at a temperature from 32° C. to 37° C.

### BACKGROUND OF THE INVENTION

### Field of the Invention

The present invention relates to a method for production of Lactobacillus plantarum.

### Background Art

MRS (deMan Rogosa Sharpe) medium is generally used for culturing Lactobacilli. This medium contains, in addition to carbon sources and nitrogen sources such as yeast extract, meat extract, glucose and peptone, sodium acetate, ammonium citrate, potassium dihydrogen phosphate, magnesium sulfate, manganese (II) sulfate, and the like.

MRS medium is often used for culturing one of lactic acid bacteria, Lactobacillus plantarum (L. plantarum). For example, R. Georgieva et al., (Biotechnology & Biotechnological Equipment 2014, 861-865) cultured L. plantarum at 37° C. using MRS medium containing glucose or lactose.

Further, in order to utilize specific strains of L. plantarum as an anti-inflammatory agent (JP2018-029491A) or an antiallergic agent (JP2007-126365A), these strains were cultured. In the former literature, unsaturated fatty acid ester was added to the medium to increase the quantity of production of IL-10 or IL-12. Moreover, in the latter literature, the bacterial cells were cultured using MRS medium.

In general, strains differ in their auxotrophic requirements, so that culture optimization is required at the strain level. For culturing L. plantarum, many examples of using MRS medium are seen. Further, when an animal for which the culture product of L. plantarum is used is a chicken, a component derived from an animal such as a meat extract cannot be added to the medium, leading to a limitation such that an MRS medium cannot be used. Accordingly, culturing of L. plantarum to be used for chickens poses a problem such that culture conditions should be studied.

### SUMMARY OF THE INVENTION

The present invention includes the following features.
[1] A method for production of Lactobacillus plantarum, comprising a step of culturing Lactobacillus plantarum in a medium comprising molasses, a yeast extract, and sucrose at a temperature from 32° C. to 37° C.
[2] The method according to [1] above, wherein the above Lactobacillus plantarum is a Lactobacillus plantarum strain (International Accession No. NITE BP-03418).
[3] The method according to [1] or [2] above, wherein in the above medium, a compounding amount of the above molasses is from 6.0% (w/w) to 13% (w/w) or more in terms of total sugar content, a compounding amount of the above yeast extract is from 0.50% (w/w) to 1.2% (w/w) or more in terms of total nitrogen, and a concentration of the above sucrose is from 3.0% (w/w) to 10% (w/w), and a pH of the above medium in neutralization culture is from 5.5 to 6.5, and, a culture time is 16 hours or longer.
[4] The method according to [3] above, wherein the compounding amount of the above molasses is from 6.7% (w/w) to 10% (w/w) or more in terms of total sugar content.
[5] The method according to [3] or [4] above, wherein the compounding amount of the above yeast extract is from 0.54% (w/w) to 1.0% (w/w) or more in terms of total nitrogen.
[6] The method according to any one of [3] to [5] above, wherein the concentration of the above sucrose is from 4.0% (w/w) to 8.0% (w/w).
[7] The method according to any one of [3] to [6] above, wherein the pH of the above medium during neutralization culture is from 5.8 to 5.9.
[8] The method according to any one of [1] to [7] above, wherein the above culture temperature is 34° C.
[9] The method according to any one of [1] to [8] above, wherein the culture time is from 20 hours to 30 hours.
[10] The method according to any one of [1] to [9] above, wherein the above medium further comprises a trace element.
[11] The method according to [10] above, wherein the above trace element comprises magnesium.
[12] The method according to any one of [1] to [10] above, wherein the above medium further comprises vitamins.
[13] The method according to any one of [1] to [12] above, further comprising a step of collecting a culture product comprising the above Lactobacillus plantarum from a culture solution after the above culture.

### BRIEF DESCRIPTION OF THE DRAWINGS

FIG. 1 shows the relationship between the culture time and the cell count when the lactic acid bacterial strain of the present invention is cultured under conditions in Table 13 (Example).

### DETAILED DESCRIPTION

The present invention will be described in more detail.

The present invention provides a method for production of Lactobacillus plantarum comprising a step of culturing Lactobacillus plantarum in a medium comprising molasses, a yeast extract and sucrose at a temperature from 32° C. to 37° C.

The above Lactobacillus plantarum may be any strain. Preferably, Lactobacillus plantarum is a Lactobacillus plantarum strain (International Accession No. NITE BP-03418).

This strain was internationally deposited at an international depositary institution under the terms of the Budapest Treaty, the NITE (National Institute of Technology and Evaluation) Patent Microorganism Depositary (#122, 2-5-8, Kazusakamatari, Kisarazu-shi, Chiba, 292-0818, Japan), on Feb. 19, 2021. The present inventors have demonstrated that this strain is useful in prevention or suppression (or improvement), etc., of necrotic enteritis in chickens.

The culture conditions for lactic acid bacteria belonging to the genus Lactobacillus should be optimized for each of the bacterial species, preferably, strains, since bacterial species and strains differ in their auxotrophic requirements.

In general, examples of essential nutrients for culturing lactic acid bacteria include carbon sources, nitrogen sources, lipids, and trace elements. However, when the culture product of Lactobacillus plantarum is utilized for birds such as chickens, a medium component derived from an animal (for example, a meat extract) cannot be used.

Under such circumstances, in the method of the present invention, molasses and sucrose are appropriate as carbon sources and a yeast extract is appropriate as a nitrogen source in a medium. The use of molasses provides an advantage that the yield (cell count; for example, about 4.0×10¹¹ or more cells/g) and the medium cost can be significantly improved.

Specific examples of the culture conditions are such that, in a medium, the compounding amount of molasses (for example, total sugar content of about 50% (w/w) to 80% (w/w)) is from about 9% (w/w) to 20% (w/w) or more, preferably about 12% (w/w) to 16% (w/w) or more, and the total sugar content is from about 4.5% (w/w) to about 16% (w/w) or more, preferably about 6.0% (w/w) to 13% (w/w) or more, the compounding amount of a yeast extract is from 0.50% (w/w) to 1.2% (w/w) or more in terms of total nitrogen, and the concentration of sucrose is from 3.0% (w/w) to 10% (w/w), and the pH of the medium in neutralization culture is from 5.5 to 6.5 and the culture time is 16 hours or longer.

Preferably, the compounding amount of molasses is from 6.7% (w/w) to 10% (w/w) or more in terms of total sugar content, the compounding amount of a yeast extract is from 0.54% (w/w) to 1.0% (w/w) or more in terms of total nitrogen, and the concentration of sucrose is from 4.0% (w/w) to 8.0% (w/w).

Further, the above medium may also contain a surfactant such as polysorbate (POLYSORBATE)™, e.g., polysorbate 80, or Tween and the like. The medium may also contain as other medium components, sodium acetate, trace elements, for example, metal salts of magnesium, manganese, zinc and the like (for example, magnesium sulfate, manganese sulfate, and zinc chloride), and others, vitamins (for example, thiamine and biotin).

Preferably, the medium contains at least polysorbate 80 in an amount of 0.3% (w/w), sodium acetate trihydrate in an amount of 0.5% (w/w), magnesium sulfateheptahydrate in an amount of 0.1% (w/w).

Regarding trace elements, a metal component(s) contained in molasses is thought to effectively act on the culturing of Lactobacillus plantarum. The reason for this is that if trace elements and vitamins are deficient, bacterial growth can be suppressed.

At the time of culturing, the pH of the medium will become acidic. Hence, an aqueous sodium hydroxide (NaOH) solution is preferably added to maintain the pH at from 5.8 to 5.9.

The culture temperature is from 28° C. to 40° C., and the preferable culture temperature is 34° C. The culture time is from 20 to 30 hours.

More preferable culture conditions are also shown in Table 13 shown later.

Culturing is performed under aerobic conditions and can be performed while stirring and maintaining the temperature and the pH at fixed levels.

Inoculation with Lactobacillus plantarum can be performed by introducing it in a proportion of, for example, 2%(v/v), into the above medium in an incubator.

During culturing, the time point of completion of culturing (for example, 20 to 30 hours) may be determined by monitoring sodium hydroxide consumption, which is proportional to lactic acid bacterial growth, and sampling the culture solution as needed over time to determine a cell count.

After completion of culturing, solid-liquid separation is performed by centrifugation to collect a fraction containing Lactobacillus plantarum as a culture product, and then the culture product is washed with water and a buffer as necessary. Furthermore, the culture product fraction can be subjected to a dry step to produce a final product. Drying can be performed by a method such as vacuum drying, lyophilization, and spray drying. With such a technique, for example, 4.0×10¹¹ or more cells/g of the cells of Lactobacillus plantarum with a water content of less than 10% can be obtained.

If further needed, dead cells of Lactobacillus plantarum may also be prepared. Dead cells can be obtained by subjecting viable cells to physical or chemical treatment such as heat treatment, formalin treatment, acid treatment, gamma ray sterilization, and disruption treatment (using a homogenizer, a blender, glass spheres, ultrasonic wave or the like).

### EXAMPLES

The present invention will be further described in detail with the following Examples, but the scope of the present invention is not limited by the Examples.

### Example 1

<Determining Optimal Conditions for Culturing Lactobacillus plantarum Strain>

### (1) Culture Method

In an incubator of a fermentor (Type: Desktop, Marubishi Bioengineering Co., Ltd., Tokyo, Japan), the Lactobacillus plantarum strain (International Accession No. NITE BP-03418) was cultured using culture conditions of different medium compositions, pH, temperatures, and times set forth below.

### (2) The Presence or the Absence of Molasses

Effects of the presence or the absence of molasses and the contents of additives on cell yield (based on NaOH consumption) are shown in Table 1, Table 2 and Table 3.

TABLE 1
Medium composition

| | | | Low | High |
|---|---|---|---|---|
| | | | concen- | concen- |
| | No | No | tration | tration |
| Medium | molasses | molasses | molasses | molasses |
| component | (w/w, %) | (w/w/, %) | (w/w, %) | (w/w, %) |
| Molasses | 0.0 | 0.0 | 6.0 | 12 |
| Yeast extract | 8.1 | 10 | 8.1 | 8.1 |
| Sucrose | 8.5 | 8.5 | 8.5 | 8.5 |
| POLYSORBATE ® | 0.3 | 0.3 | 0.3 | 0.3 |
| 80 | | | | |
| CH₃COONa•3H₂O | 0.5 | 0.5 | 0.5 | 0.5 |
| MgSO₄•7H₂O | 0.1 | 0.1 | 0.1 | 0.1 |
| Water | 83 | 83 | 81 | 77 |

TABLE 2
Effects of the presence or the absence of molasses and the
amount of molasses added on cell yield (NaOH consumption)

| | Total nitrogen | NaOH consumption |
|---|---|---|
| Medium component | (%) | (g) |
| No molasses 1 | 0.84 | 254 |
| No molasses 2 | 1.20 | 256 |
| Low concentration | 0.84 | 313 |
| molasses | | |
| High concentration | 0.84 | 321 |
| molasses | | |

As shown in Table 2, addition of molasses resulted in a sharp increase in NaOH consumption. It was considered that a trace component in molasses contributed to the yield.

TABLE 3
Effects of the amount of molasses added
on cell yield (NaOH consumption)

| Molasses | Total nitrogen | NaOH consumption |
|---|---|---|
| (%) | (%)* | (g) |
| 6.0 | 0.84 | 304 |
| 9.3 | 0.84 | 313 |
| 12 | 0.84 | 321 |
| 14 | 0.84 | 306 |

*Total nitrogen was fixed at 0.84% to conduct test.

As shown in Table 3, the maximum amount (%) of molasses added is 12%.

In old production methods, molasses is not used as a medium component. However, in the production methods of this invention, addition of molasses resulted in increased cell counts (yields) (Table 4).

TABLE 4
Comparison of cell count based on the
presence or the absence of molasses

| | Yielded cell | Medium | NaOH | Total |
|---|---|---|---|---|
| | count | amount | consumption* | cell count |
| | (cells/mL) | (kg) | (kg) | (cells/batch) |
| Old | 6.65 × 10⁹ | 2.3 | 0.2067 | 1.67 × 10¹³ |
| production | | | | |
| method 1 | | | | |
| Old | 7.25 × 10⁹ | 2.3 | 0.1972 | 1.81 × 10¹³ |
| production | | | | |
| method 2 | | | | |
| Method 1 of | 2.03 × 10¹⁰ | 2.3 | 0.3056 | 5.29 × 10¹³ |
| this invention | | | | |
| Method 2 of | 1.67 × 10¹⁰ | 2.3 | 0.3147 | 4.37 × 10¹³ |
| this invention | | | | |

*NaOH consumption: Can be used for evaluation of cell amount at the time of neutralization culture. Yields are compared based on NaOH consumption and cell count in neutralization culture.

The test results in the production line are as follows (Table 5).

TABLE 5
Comparison of cell count in production line

| | Amount | Total cell | Dry cell | Dry cell |
|---|---|---|---|---|
| | added | count/batch | powder | concentration |
| Method | (kg) | (cells) | (kg) | (cells/g) |
| Old production | 2800 | 1.89 × 10¹⁶ | 34.7 | 5.44 × 10¹¹ |
| method | | | | |
| (measured | | | | |
| value) | | | | |
| Method of this | 2800 | 5.20 × 10¹⁶ | 53.5 | 9.92 × 10¹¹ |
| invention | | | | |
| (measured | | | | |
| value) | | | | |

### (3) Amount of Yeast Extract Added

With the medium composition shown in Table 6, a culture temperature of 34° C., stirring at 100 rpm, a medium capacity of 2.3 kg, and pH 5.9, culturing was performed under conditions of neutralization culture and the culture time of 27 hours.

TABLE 6
Medium composition

| | Low amount | Moderate amount | High amount |
|---|---|---|---|
| | added | added | added |
| Medium component | (w/w, %) | (w/w, %) | (w/w, %) |
| Yeast extract | 7.0 | 10 | 12 |
| Sucrose | 8.5 | 8.5 | 8.5 |
| POLYSORBATE ® 80 | 0.3 | 0.3 | 0.3 |
| CH₃COONa•3H₂O | 0.5 | 0.5 | 0.5 |
| MgSO₄•7H₂O | 0.1 | 0.1 | 0.1 |
| Water | 84 | 81 | 79 |

The results are shown in Table 7.

TABLE 7
Effects of the amount of yeast extract
added on cell yield (NaOH consumption)

| Yeast extract | Total nitrogen | NaOH consumption |
|---|---|---|
| (w/w, %) | (%) | (g) |
| 7.0 | 0.84 | 239 |
| 10 | 1.20 | 256 |
| 12 | 1.44 | 252 |

When the amount of the carbon source was sufficient and a yeast extract supplied a nitrogen source and the trace amounts of minerals, the total nitrogen was 1.2% and the growth of the lactic acid bacterial strain of this invention reached a plateau. The results indicate that the trace components may be a growth rate-limiting factor.

The effects on the growth of the lactic acid bacterial strain of this invention were examined when the concentration of molasses was fixed at 12% and the amount of a yeast extract added was decreased. The results are shown in Table 8.

TABLE 8
Effects of the amount of yeast extract
added on cell yield (NaOH consumption)

| | Total nitrogen | NaOH consumption |
|---|---|---|
| Yeast extract | (%) | (g) |
| High amount added | 0.84 | 323 |
| Moderate amount added | 0.54 | 302 |
| Low amount added | 0.24 | 229 |

Table 8 shows that since a significant improvement in yield cannot be expected even when the total nitrogen exceeds 0.54%, efficiency is excellent with the total nitrogen of a yeast extract of 0.54%. However, since molasses is a natural raw material, the composition of micronutrients may vary depending on lots. Since the mineral composition of molasses changes with the season and other factors, the amount (%) of molasses added should be increased by about 2%. This means that the amount of Molasses added to achieve stable production is 14%.

### (4) Effects of the Amount of Sucrose Added

The amount of sucrose needed was examined by fixing the amount (%) of molasses added at 14%. The results are shown in Table 9.

TABLE 9
Effects of the presence or the absence of sucrose and the
amount of sucrose added on cell yield (NaOH consumption)

| Sucrose | NaOH consumption | Cell count |
|---|---|---|
| (w/w, %) | (g) | (cells/mL) |
| 0 | 291 | 1.21 × 10¹⁰ |
| 4 | 327 | 1.79 × 10¹⁰ |
| 8 | 330 | 1.78 × 10¹⁰ |

Table 9 shows that the amount (%) of sucrose added of 4% is appropriate.

### (5) Effects of Temperatures

The effects of culture temperatures of 28° C., 34° C., and 40° C. on the growth of the lactic acid bacterial strain of this invention are examined. The results are shown in Table 10.

TABLE 10
Effects of temperature on cell yield (NaOH consumption)

| Culture temperature | NaOH consumption |
|---|---|
| (° C.) | (g) |
| 28 | 88.72 |
| 34 | 114.0 |
| 40 | 94.81 |

The culture temperature of 34° C. is appropriate.

### (6) Effects of pH

The effects of pH 5.0, 5.8, and 6.5 in neutralization culture on the growth of the lactic acid bacterial strain of this invention were examined. The results are shown in Table 11.

TABLE 11
Effects of pH on cell yield (NaOH consumption)

| | NaOH consumption |
|---|---|
| pH | (g) |
| 5.0 | 98.66 |
| 5.8 | 176.7 |
| 6.5 | 148.1 |

As shown in Table 11, the optimal pH for neutralization culture is around 5.8.

### (7) Effects Due to Culture Time

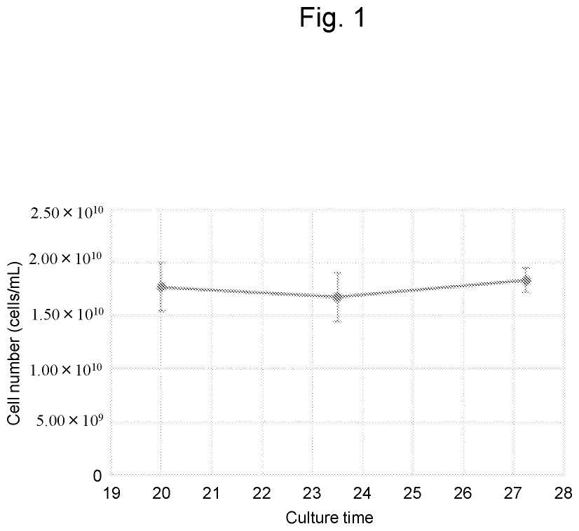

The relationship between culture time and cell count was examined when the lactic acid bacterial strain of this invention was cultured under the conditions in Table 13 (described below). As a result, it was revealed that even 20 hours or longer of culturing did not result in a remarkable change in cell count (Table 12, FIG. 1).

TABLE 12
Effects of culture time on cell count

| Culture time | Cell count |
|---|---|
| (hr) | (cells/mL) |
| 20 | 1.55 × 10¹⁰ |
| 23.5 | 1.54 × 10¹⁰ |
| 27.2 | 1.56 × 10¹⁰ |

### (8) Optimal Conditions

Based on the above results, optimal conditions are shown in Table 13.

TABLE 13
Optimal conditions of medium components

| | Optimal conditions |
|---|---|
| Medium component | (w/w, %) |
| Molasses | 14* |
| Yeast extract | 5.19 |
| (Total nitrogen) | (0.54) |
| Sucrose | 4.0 |
| POLYSORBATE ® 80 | 0.3 |
| CH₃COONa•3H₂O | 0.5 |
| MgSO₄•7H₂O | 0.1 |
| Water | 76 |

*Total sugar content of 6.7%(w/w)

Furthermore, the optimal culture temperature, the optimal pHs and the optimal culture time were as follows.

The optimal culture temperature is 34° C.

Regarding the optimal pH, neutralization culture is performed at a pH from 5.8 to 5.9.

The optimal culture time is 20 hours or longer.

### INDUSTRIAL APPLICABILITY

According to this invention, optimal culture conditions for one of lactic acid bacteria, Lactobacillus plantarum, were determined, and thus the yield of the above lactic acid bacteria can be efficiently increased in the production of the lactic acid bacteria.

What is claimed is:

1. A method for production of Lactobacillus plantarum, comprising a step of culturing Lactobacillus plantarum in a medium comprising molasses, a yeast extract, and sucrose, at a temperature from 32° C. to 37° C.

2. The method according to claim 1, wherein the Lactobacillus plantarum is a Lactobacillus plantarum strain (International Accession No. NITE BP-03418).

3. The method according to claim 1, wherein in the medium,
(i) the molasses is present at a concentration in a range from 6.0% (w/w) to 13% (w/w) or more in terms of total sugar content,
(ii) the yeast extract is present at a concentration in a range from 0.50% (w/w) to 1.2% (w/w) or more in terms of total nitrogen, and
(iii) the sucrose is present at a concentration in a range from 3.0% (w/w) to 10% (w/w),
wherein the medium during neutralization culture has a pH in a range from 5.5 to 6.5, and
wherein a culture time is 16 hours or longer.

4. The method according to claim 3, wherein the molasses is present at a concentration in a range from 6.7% (w/w) to 10% (w/w) or more in terms of total sugar content.

5. The method according to claim 3, wherein the yeast extract is present at a concentration in a range from 0.54% (w/w) to 1.0% (w/w) or more in terms of total nitrogen.

6. The method according to claim 3, wherein the concentration of the sucrose is from 4.0% (w/w) to 8.0% (w/w).

7. The method according to claim 3, wherein the pH of the medium during neutralization culture is from 5.8 to 5.9.

8. The method according to any one of claims 1 to 3, wherein the temperature is 34° C.

9. The method according to claim 3, wherein the culture time is from 20 hours to 30 hours.

10. The method according to any one of claims 1 to 3, wherein the medium further comprises a trace element.

11. The method according to claim 10, wherein the trace element comprises magnesium.

12. The method according to any one of claims 1 to 3, wherein the medium further comprises vitamins.

13. The method according to any one of claims 1 to 3, further comprising a step of collecting a culture product comprising the Lactobacillus plantarum from a culture solution after the culture.
